(19) United States
(12) Patent Application Publication
(10) Pub. No.: US 2022/0084835 A1
(43) Pub. Date: Mar. 17, 2022

(54) PLASMA ETCHING METHOD AND PLASMA ETCHING APPARATUS

(71) Applicant: Kioxia Corporation, Tokyo (JP)

(72) Inventors: Shuichi KUBOI, Yokohama (JP); Daiki IINO, Yokohama (JP); Hiroyuki FUKUMIZU, Yokohama (JP)

(73) Assignee: Kioxia Corporation, Tokyo (JP)

(21) Appl. No.: 17/346,810

(22) Filed: Jun. 14, 2021

(30) Foreign Application Priority Data
Sep. 11, 2020 (JP) 2020-152885

(51) Int. Cl. H01L 21/311; H01J 37/32; C09K 13/00; C07C 21/22; C07C 21/215; C07C 21/20; C07C 23/06; C07C 23/26

(52) U.S. Cl. CPC H01L 21/31116; H01L 21/31144; H01J 37/3244; C09K 13/00; C07C 21/22; C07C 21/215; C07C 2602/18; C07C 23/06; C07C 23/26; H01J 2237/334; H01J 37/32082; C07C 2601/04; C07C 21/20

(57) ABSTRACT

A plasma etching method according to an embodiment is a method for etching a silicon-containing film by using plasma of a fluorocarbon gas. The fluorocarbon gas includes at least one selected from a first fluorocarbon which has a main chain of six or more carbons bonded in a linear manner, the main chain having a structure of single bond and double bond alternately joined, a second fluorocarbon which has a main chain of six or more carbons bonded in a linear manner, the main chain having a structure of single bond and triple bond alternately joined, and a third fluorocarbon which has a main chain of five or more carbons bonded in a linear manner, the main chain having a structure which includes double bond and triple bond.

### CROSS REFERENCE TO RELATED APPLICATION

This application is based upon and claims the benefit of priority from Japanese Patent Application No. 2020-152885, filed on Sep. 11, 2020; the entire contents of which are incorporated herein by reference.

### FIELD

Embodiments described herein relate generally to a plasma etching method and a plasma etching apparatus.

### BACKGROUND

In a manufacturing process of a semiconductor device, plasma etching is performed to form a contact hole, a via hole, a trench (groove) and so on in a silicon-containing film such as a silicon oxide film formed on a semiconductor substrate or the like. In the manufacturing process of the semiconductor device as above, precise control of a processing shape, especially vertical processing of a side wall of the contact hole or the like is important to ensure electrical performance or the like of the semiconductor device. For example, a recent three-dimensional structure device has a hole with a large aspect ratio. In forming such a hole with a large aspect ratio by plasma etching, it is desired to increase a hole etching rate per hour and a processing selection ratio with respect to an etching mask.

### BRIEF DESCRIPTION OF THE DRAWINGS

FIG. 1 is a cross-sectional view illustrating a first example of a plasma etching apparatus of an embodiment.

FIG. 2 is a cross-sectional view illustrating a second example of the plasma etching apparatus of the embodiment.

FIG. 3 is a diagram illustrating a first example of a fluorocarbon gas used for a plasma etching method of an embodiment.

FIG. 4 is a diagram illustrating a second example of the fluorocarbon gas used for the plasma etching method of the embodiment.

FIG. 5 is a diagram illustrating a third example of the fluorocarbon gas used for the plasma etching method of the embodiment.

FIG. 6 is a table illustrating a C/F ratio of the fluorocarbon gas used for the plasma etching method of the embodiment.

FIG. 7 is a diagram illustrating a fluorocarbon gas as a comparative example.

FIG. 8 is a chart illustrating a hole etching rate and a mask etching rate of the fluorocarbon gas used for the plasma etching method of the embodiment.

FIG. 9 is a chart illustrating a processing selection ratio with respect to an etching mask of the fluorocarbon gas used for the plasma etching method of the embodiment.

FIG. 10 is a chart illustrating a relation of the processing selection ratio with respect to the etching mask to the hole etching rate of the fluorocarbon gas used for the plasma etching method of the embodiment.

FIG. 11 is a diagram for explaining the hole etching rate, the mask etching rate, and the processing selection ratio with respect to the etching mask in FIG. 8 to FIG. 10.

### DETAILED DESCRIPTION

A plasma etching method according to an aspect of embodiments includes etching a silicon-containing film by using plasma of a fluorocarbon gas, wherein the fluorocarbon gas includes a fluorocarbon which has a main chain of six or more carbons bonded in a linear manner, the main chain having a structure of single bond and double bond alternately joined or single bond and triple bond alternately joined.

A plasma etching method according to another aspect of the embodiments includes etching a silicon-containing film by using plasma of a fluorocarbon gas, wherein the fluorocarbon gas includes a fluorocarbon which has a main chain of five or more carbons bonded in a linear manner, the main chain having a structure which includes double bond and triple bond.

Hereinafter, a plasma etching method and a plasma etching apparatus of the embodiments will be described with reference to the drawings. In respective embodiments, substantially the same constituent portions will be denoted by the same reference numeral and explanation thereof may be partially omitted. The drawings are schematic, and a relation between a thickness and a planer dimension, a thickness ratio of the respective parts, and so on may be different from actual ones. A term indicating a direction such as an upper or lower direction in the explanation indicates a relative direction when a later-described plasma etching surface (processing surface) of a substrate is faced upward, unless otherwise specified, and is sometimes different from an actual direction based on a gravitational acceleration direction.

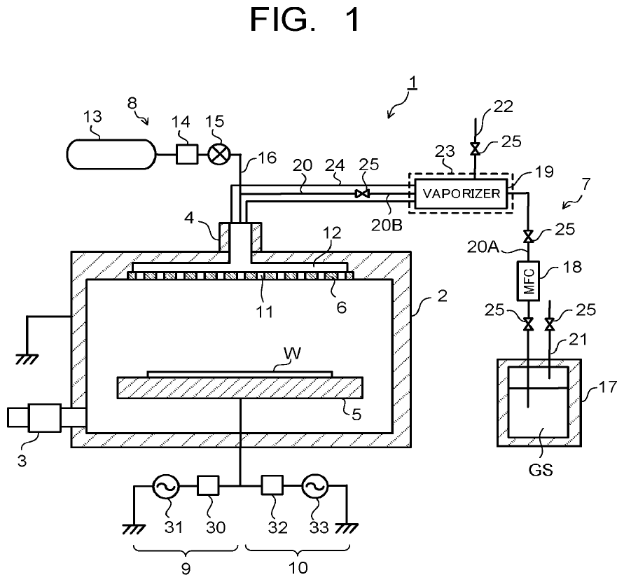

FIG. 1 is a cross-sectional view illustrating a first example of the plasma etching apparatus according to the embodiment. A plasma etching apparatus 1 illustrated in FIG. 1 is a reactive ion etching (RIE) apparatus of parallel plate type, and has a chamber 2, an exhaust port 3, a process gas introduction port 4, a lower electrode (substrate electrode) 5, an upper electrode (counter electrode) 6, a first process gas introduction unit 7, a second process gas introduction unit 8, a first power supply system 9, and a second power supply system 10.

The chamber 2 is provided with the exhaust port 3 and the process gas introduction port 4. The exhaust port 3 is connected to not-illustrated pressure regulating valve, exhaust pump, and so on. A gas in the chamber 2 is exhausted from the exhaust port 3, resulting in that the inside of the chamber 2 is kept at high vacuum. When introducing a process gas from the process gas introduction port 4, by balancing a flow rate of the gas which flows from the process gas introduction port 4 with a flow rate of the gas which flows out of the exhaust port 3, it is possible to keep a pressure inside the chamber 2 to a certain vacuum pressure.

To the process gas introduction port 4 of the chamber 2, the first process gas introduction unit 7 and the second process gas introduction unit 8 are connected. The upper electrode 6 has a plurality of gas discharge ports 11. The chamber 2 is provided with a gas introduction space 12 facing the plurality of gas discharge ports 11 of the upper electrode 6, in a manner that the gas introduction space 12 is connected to the process gas introduction port 4. The first process gas introduction unit 7 has a mechanism of vaporizing a process gas raw material in a liquid state at room temperature and introducing the vaporized process gas raw material into the chamber 2. The second process gas introduction unit 8 is for introducing a process gas in a gaseous state at room temperature into the chamber 2.

The second process gas introduction unit 8 has a gas supply source 13, a mass flow controller 14 which controls a gas flow rate, an opening/closing valve 15, and a pipe 16. One end of the pipe 16 is connected to the gas supply source 13, and the other end of the pipe 16 is connected to the process gas introduction port 4. As the process gas in the gaseous state at room temperature, for example, there are used a rare gas such as He, Ar, Kr, or Xe, a gas such as N₂, O₂, H₂, CO, NF₃, SF₆, or CH₄, a general CₓF_{y} gas (fluorocarbon gas) such as CF₄, C₄F₆, or C₄F₈, and a CₓH_{y}F_{z} gas (hydrofluorocarbon gas) such as CHF₃, CH₂F₂, or CH₃F.

The first process gas introduction unit 7 has a raw material tank 17 which accommodates a liquid process gas raw material GS, a liquid flow rate controller 18, a vaporizer 19 as a vaporization mechanism which vaporizes the liquid process gas raw material GS, and a pipe 20 which connects these raw material tank 17, liquid flow rate controller 18, and vaporizer 19. One end of the pipe 20 is opened to the inside of the raw material tank 17, and the other end of the pipe 20 is connected to the process gas introduction port 4. To the raw material tank 17, an inert gas supply line 21 is connected. The liquid flow rate controller 18 is installed in a first pipe 20A which feeds the process gas raw material GS to the vaporizer 19.

To the vaporizer 19, there is connected a carrier gas supply line 22 which supplies a carrier gas for feeding a vaporized component (process gas) of the process gas raw material GS into the chamber 2. A periphery of the vaporizer 19 is covered by a heat insulating material 23. Further, in order to prevent the vaporized component of the process gas raw material GS from liquefying in the pipe 20, a heater 24 is provided in a periphery of a second pipe 20B from the vaporizer 19 to the process gas introduction port 4. Each of the pipe 20, the inert gas supply line 21, and the carrier gas supply line 22 is provided with an opening/closing valve 25 according to a necessary place.

In the first process gas introduction unit 7, as a result that an inert gas is supplied from the inert gas supply line 21 to the raw material tank 17, the process gas raw material GS is fed to the vaporizer 19 via the liquid flow rate controller 18. The liquid process gas raw material GS, whose flow rate is controlled by the liquid flow rate controller 18, is vaporized by the vaporizer 19. Since the flow rate of the liquid process gas raw material GS is controlled by the liquid flow rate controller 18, the vaporized component of the process gas raw material GS vaporized by the vaporizer 19 is fed into the chamber 2 via the process gas introduction port 4, at a predetermined gas flow rate. The liquid process gas raw material GS and the vaporized component thereof will be described later in detail.

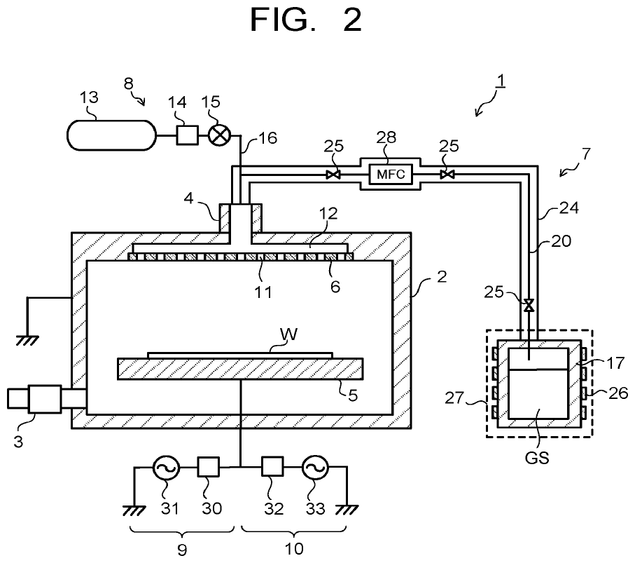

A mechanism of vaporizing the process gas raw material GS is not limited to a configuration using the vaporizer 19 illustrated in FIG. 1. FIG. 2 is a cross-sectional view illustrating a second example of the plasma etching apparatus of the embodiment. As illustrated in FIG. 2, the plasma etching apparatus may have a vaporization mechanism which vaporizes a liquid process gas raw material GS by directly heating a raw material tank 17 which accommodates the liquid process gas raw material GS. In other words, in a first process gas introduction unit 7 illustrated in FIG. 2, a heater 26 is provided in a periphery of the raw material tank 17 which accommodates the liquid process gas raw material GS, and besides, a periphery of the raw material tank 17 and the heater 26 is covered by a heat insulating material 27. A pipe 20 from the raw material tank 17 to a process gas introduction port 4 is provided with a gas flow rate controller 28. A heater 24 is provided in a periphery of the pipe 20 and the gas flow rate controller 28.

In the first process gas introduction unit 7 illustrated in FIG. 2, the raw material tank 17 which accommodates the liquid process gas raw material GS is directly heated by the heater 26. The liquid process gas raw material GS heated by the heater 26 is vaporized, and a resulting vaporized component is fed to the pipe 20. A flow rate of the vaporized component of the process gas raw material GS is controlled by the gas flow rate controller 28, and the vaporized component is fed in this state into the chamber 2 via the process gas introduction port 4.

In the chamber 2, the lower electrode 5 is provided as a substrate electrode which is vertically movable and which also functions as a mounting table (holding part) to mount a substrate such as a semiconductor wafer W thereon. It is configured such that on an upper part of the lower electrode 5, a not-illustrated electrostatic chuck is provided, so that the semiconductor wafer W can be held on the lower electrode 5. Above the lower electrode 5, the upper electrode 6 which also functions as a shower head for process gas discharge is placed, as a counter electrode, at a position separating the gas introduction space 12 from a processing space where etching process of the semiconductor wafer W is performed. The upper electrode 6 is provided with the plurality of gas discharge ports 11 to supply the process gas from the gas introduction space 12 to the processing space of the semiconductor wafer W. The chamber 2 is grounded.

To the lower electrode 5 as the substrate electrode, the first power supply system 9 and the second power supply system 10 are connected. The first power supply system 9 has a matching device 30 and a first high-frequency power supply 31, and the second power supply system 10 has a matching device 32 and a second high-frequency power supply 33. The first high-frequency power supply 31 is a power supply which outputs a first high-frequency voltage (Va) for ionizing the process gas to generate plasma, and the output first high-frequency voltage (Va) is applied to the lower electrode 5. The second high-frequency power supply 33 is a power supply which outputs a second high-frequency voltage (Vb) for attracting ions from plasma to the semiconductor wafer W, a frequency of the second high-frequency voltage (Vb) being lower than that of the first high-frequency voltage (Va), and the output second high-frequency voltage (Vb) is applied to the lower electrode 5. Both the voltage Va and the voltage Vb are generally referred to as high-frequency voltages, but in order to explain a difference between the respective frequencies, the first high-frequency voltage (Va) is referred to as an RF high-frequency voltage, and the second high-frequency voltage (Vb) is referred to as an RF low-frequency voltage, for the sake of convenience.

The RF high-frequency voltage (Va) output by the first high-frequency power supply 31 is preferably 27 MHz or more for increasing power of generating plasma, and is preferably 100 MHz, 60 MHz, 40 MHz, 27 MHz, or the like, for example. The RF low-frequency voltage (Vb) output by the second high-frequency power supply 33 is preferably 3 MHz or less for increasing ion attraction from plasma, and is preferably 3 MHz, 2 MHz, 400 kHz, 100 kHz, or the like, for example. A voltage between an upper peak and a lower peak of the RF low-frequency voltage (Vb) which is applied from the second high-frequency power supply 33 to the lower electrode 5 is preferably 1000 V or more.

At the same time as the process gas is introduced from the first process gas introduction unit 7 into the chamber 2 and, if necessary, the process gas is introduced from the second process gas introduction unit 8 into the chamber 2, the RF high-frequency voltage (Va) and the RF low-frequency voltage (Vb) are applied to the lower electrode 5 from the aforementioned first high-frequency power supply 31 and second high-frequency power supply 33, respectively, resulting in that plasma is generated between the lower electrode 5 and the upper electrode 6. In other words, as a result that the RF high-frequency voltage (Va) from the first high-frequency power supply 31 and the RF low-frequency voltage (Vb) from the second high-frequency power supply 33 are superposed and applied to the lower electrode 5, the process gas is ionized to form plasma of the process gas between the lower electrode 5 and the upper electrode 6, and simultaneously, ions are attracted toward the lower electrode 5 side.

Next, a plasma etching method of a semiconductor wafer W by using the above-described plasma etching apparatus 1 will be described. In a plasma etching method of an embodiment, a substrate such as a semiconductor wafer W to be etched is first mounted on the lower electrode 5. The semiconductor wafer W to be etched has a silicon-containing film such as a silicon oxide film (SiO film) or a silicon nitride film (SiN film) formed on a semiconductor film or a metal film which includes at least one selected from a group consisting of silicon, tungsten, aluminum, titanium, molybdenum, and tantalum. An etching mask is formed on the semiconductor wafer W having such a silicon-containing film of SiO film or SiN film, and the etching mask is patterned to form an opening, and then plasma etching process is performed, to thereby form a hole portion such as a contact hole on the silicon-containing film in accordance with the opening of the etching mask.

When forming the contact hole or the like in the silicon-containing film, the process gas is introduced from the first process gas introduction unit 7 into the chamber 2, and simultaneously, the RF high-frequency voltage (Va) and the RF low-frequency voltage (Vb) are applied from the first high-frequency power supply 31 and the second high-frequency power supply 33, respectively, to the lower electrode 5 on which the semiconductor wafer W with the etching mask formed thereon is mounted. Plasma is generated between the lower electrode 5 and the upper electrode 6, and ions in the plasma are attracted to the semiconductor wafer W, to thereby perform etching process on the silicon-containing film. The etching process of the silicon-containing film is performed, for example, on the SiO film. The silicon-containing film to be plasma-etched is not limited to a single film of SiO film, but may also be a stacked film of SiO film and SiN film. In the etching process of the silicon-containing film, it is possible to selectively process the silicon-containing film based on a difference in etching rate between the silicon-containing film and the aforementioned semiconductor film or metal film.

In the plasma etching process of the silicon-containing film described above, as the process gas raw material GS which is accommodated in the raw material tank 17 of the first process gas introduction unit 7, there is used a raw material which includes at least one selected from a first fluorocarbon which has a main chain of six or more carbons bonded in a linear manner, the main chain having a structure of single bond and double bond alternately joined, a second fluorocarbon which has a main chain of six or more carbons bonded in a linear manner, the main chain having a structure of single bond and triple bond alternately joined, and a third fluorocarbon which has a main chain of five or more carbons bonded in a linear manner, the main chain having a structure which includes double bond and triple bond. The first, second, and third fluorocarbons are in a liquid state at room temperature, so that the mechanism of vaporizing the liquid process gas raw material GS as illustrated in each of FIG. 1 and FIG. 2 is used.

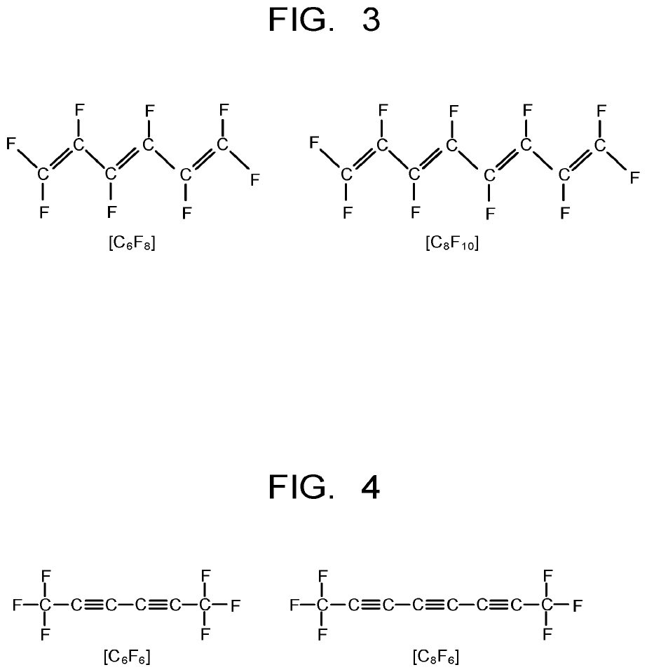

A concrete example of the first fluorocarbon is illustrated in FIG. 3. C₆F₈, whose structural formula is illustrated in FIG. 3, has a main chain of six carbons bonded in a linear manner, and the main chain has a structure of single bond and double bond alternately joined. C₈F₁₀, whose structural formula is illustrated in FIG. 3, has a main chain of eight carbons bonded in a linear manner, and the main chain has a structure of single bond and double bond alternately joined. The first fluorocarbon preferably has a composition, regarding carbon and fluorine, represented by Cₓ₁F_{y1} (in the formula, x1 and y1 are numbers satisfying x1>6 and y1>x1+2).

A concrete example of the second fluorocarbon is illustrated in FIG. 4. C₆F₆, whose structural formula is illustrated in FIG. 4, has a main chain of six carbons bonded in a linear manner, and the main chain has a structure of single bond and triple bond alternately joined. C₈F₆, whose structural formula is illustrated in FIG. 4, has a main chain of eight carbons bonded in a linear manner, and the main chain has a structure of single bond and triple bond alternately joined. The second fluorocarbon preferably has a composition, regarding carbon and fluorine, represented by Cₓ₂F_{y2} (in the formula, x2 and y2 are numbers satisfying x2>6 and y2>6).

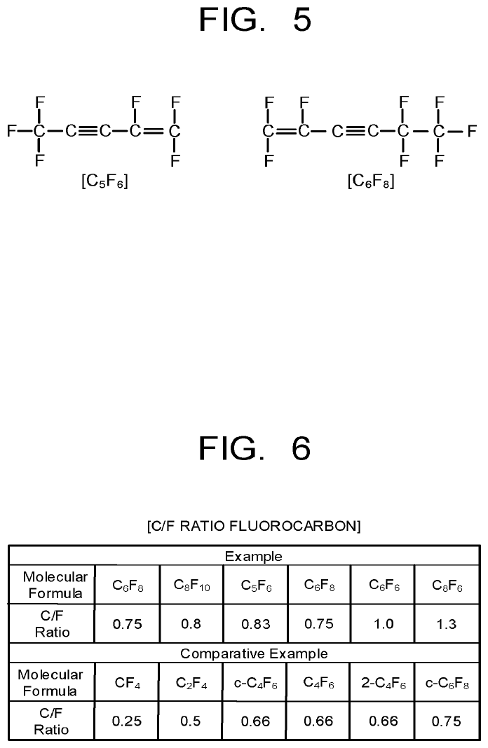

A concrete example of the third fluorocarbon is illustrated in FIG. 5. C₅F₆, whose structural formula is illustrated in FIG. 5, has a main chain of five carbons bonded in a linear manner, and the main chain has a structure which includes double bond and triple bond. Concretely, the main chain has a portion formed of double bond and triple bond joined via single bond. C₆F₈, whose structural formula is illustrated in FIG. 5, has a main chain of six carbons bonded in a linear manner, and the main chain has a structure which includes double bond and triple bond. Concretely, the main chain has a portion formed of double bond and triple bond joined via single bond, similarly to C₅F₆. Of the two carbons constituting the triple bond, the carbon on a side opposite to the carbon of a side of the double bond can be joined to a fluorine-containing alkyl group such as a —CF₃ group or a —C₂F₅ group. The third fluorocarbon preferably has a composition, regarding carbon and fluorine, represented by Cₓ₃F₃ (in the formula, x3 and y3 are numbers satisfying x3≥5 and y3≥6).

As described above, the process gas introduced from the first process gas introduction unit 7 into the chamber 2 preferably includes at least one selected from a group consisting of C₅F₆, C₆F₆, C₆F₈, C₈F₆, and C₈F₁₀. Note that the first, second, and third fluorocarbons are not limited to perfluorocarbons, but may also be compounds in which a part thereof is substituted by hydrogen or oxygen. The aforementioned fluorocarbon may be one represented by a composition formula which further includes one or more of H or O, in addition to the composition represented by CₓF_{y}.

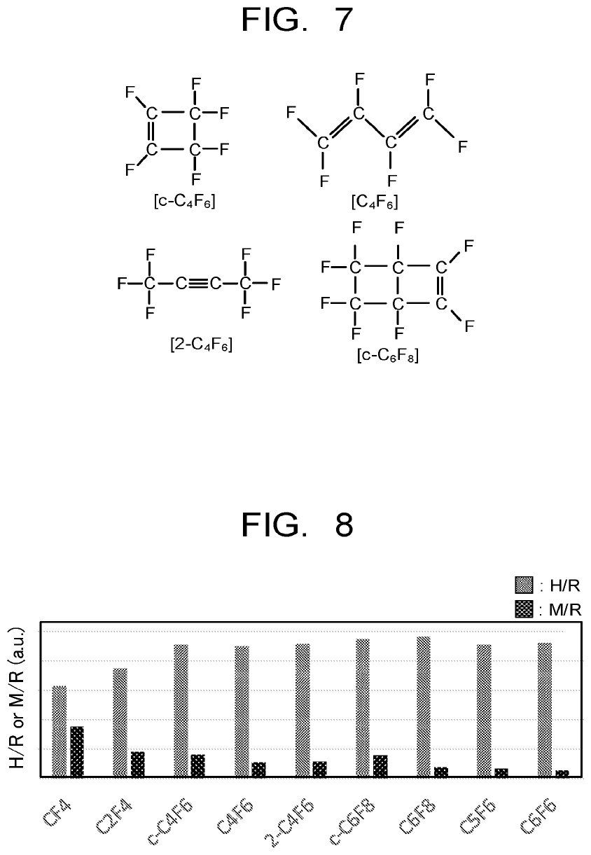

A ratio (C/F ratio) of carbon (C) to fluorine (F) of each of the first, second and third fluorocarbons is illustrated in a table of FIG. 6. C/F ratios of fluorocarbons of comparative examples are also illustrated together in the table of FIG. 6. Parts of structures of the fluorocarbons of the comparative examples are illustrated in FIG. 7. As illustrated in the table of FIG. 6, the C/F ratios of the first, second, and third fluorocarbons are each equal to or larger than the C/F ratios of the comparative examples, that is, for example, equal to or larger than 0.75. In other words, since a C amount per molecule is large, a deposition property during plasma etching process is high. Therefore, the processing selection ratio of the silicon-containing film with respect to the etching mask can be improved.

Since the first, second, and third fluorocarbons include carbon-carbon triple bond or carbon-carbon double bond, which is difficult to dissociate, in the main chain, ions with a large molecular weight which include carbon and fluorine are likely to be generated in the plasma. This improves an etching yield, bringing about an excellent etching effect. Further, as described above, the first, second, and third fluorocarbons are excellent in processing selection ratio with respect to the etching mask, so that plasma application power can be increased while the processing selection ratio with respect to the etching mask is maintained. Therefore, a hole etching rate can be enhanced in etching a silicon-containing film such as a silicon oxide film (SiO) which is formed on a semiconductor wafer W.

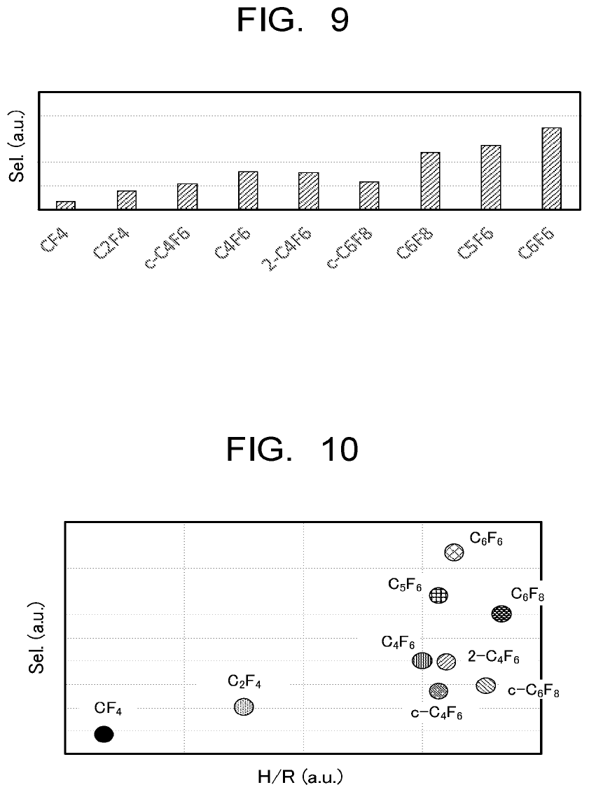

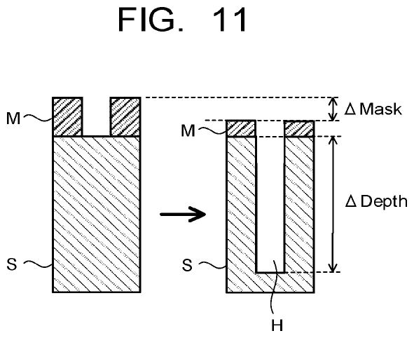

FIG. 8 illustrates a hole etching rate (H/R) and a mask etching rate (M/R) of a vaporized component (fluorocarbon gas) of each of the first, second, and third fluorocarbons. FIG. 9 illustrates a processing selection ratio (Sel.) with respect to the etching mask, of the vaporized component of each of the first, second, and third fluorocarbons. Further, FIG. 10 illustrates a relation of the processing selection ratio (Sel.) with respect to the etching mask to the hole etching rate (H/R) of the vaporized component of each of the first, second, and third fluorocarbons. As illustrated in FIG. 11, the hole etching rate (H/R) is a value obtained by dividing a depth of a hole H (ΔDepth) by a processing time (time), and the mask etching rate (M/R) is a value obtained by dividing a reduced thickness of a mask M (ΔMask) during processing by the processing time (time). The processing selection ratio (Sel.) with respect to the etching mask is a value obtained by dividing the depth of the hole H (ΔDepth) by the reduced thickness of the mask M (ΔMask). In FIG. 11, S indicates a silicon-containing film to be etched and M indicates an etching mask.

FIG. 8, FIG. 9, and FIG. 10 illustrate properties of fluorocarbon gases, that is, of C₆F₈ gas being the example of the first fluorocarbon, C₆F₆ gas being the example of the second fluorocarbon, and C₅F₆ gas being the example of the third fluorocarbon gas. FIG. 8, FIG. 9, and FIG. 10 also illustrate properties of CF₄ gas, C₂F₄ gas, c-C₄F₆ gas, C₄F₆ gas, 2-C₄F₆ gas, and c-C₆F₈ gas, whose molecular structures are illustrated in FIG. 7, for comparison.

As illustrated in FIG. 8 and FIG. 9, it can be seen that the vaporized components (fluorocarbon gases) of the first, second, and third fluorocarbons have large hole etching rates (H/R) and small mask etching rates (M/R). Regarding the vaporized components of the first, second, and third fluorocarbons, there is an example where a hole etching rate (H/R) is almost equal to that of c-C₆F₈ gas, but a mask etching rate (M/R) is clearly smaller than that of c-C₆F₈ gas. Thus, as illustrated in FIG. 9, it can be seen that the vaporized components of the first, second, and third fluorocarbons are excellent in processing selection ratio (Sel.) with respect to the etching mask. Further, as illustrated in FIG. 10, it can be seen that the vaporized components of the first, second, and third fluorocarbons are superior to the vaporized components of the comparative examples in terms of a relation of the processing selection ratio (Sel.) with respect to the etching mask to the hole etching rate (H/R).

As illustrated in FIG. 8, FIG. 9, and FIG. 10, according to the fluorocarbon gases constituted by the vaporized components of the first, second, and third fluorocarbons, a good hole etching rate can be obtained and, in addition, the processing selection ratio with respect to the etching mask can be enhanced. Thereby, it is possible to increase the plasma application power while maintaining a state of the etching mask or the like. Therefore, it becomes possible to form a contact hole or the like with a large aspect ratio accurately and efficiently in a SiO film or a stacked film of SiO film and SiN film.

Further, the fluorocarbon gases constituted by the vaporized components of the first, second, and third fluorocarbons have large etching rates on the silicon-containing film compared with an etching rate on a metal film such as tungsten (W). Therefore, in etching the silicon-containing film formed on the metal film such as a W film, the silicon-containing film can be selectively processed.

Note that the configurations of the aforementioned respective embodiments can be mutually combined to be carried out, and a part thereof can be substituted. Here, while certain embodiments of the present invention have been described, these embodiments have been presented by way of example only, and are not intended to limit the scope of the inventions. The novel embodiments described herein may be embodied in a variety of other forms; furthermore, various omissions, substitutions, changes, and so on may be made therein without departing from the spirit of the inventions. The accompanying claims and their equivalents are intended to cover such forms or modifications as would fall within the scope and spirit of the inventions.

What is claimed is:

1. A plasma etching method, comprising:
etching a silicon-containing film by using plasma of a fluorocarbon gas, wherein
the fluorocarbon gas includes a fluorocarbon which has a main chain of six or more carbons bonded in a linear manner, the main chain having a structure of single bond and double bond alternately joined or single bond and triple bond alternately joined.

2. The method according to claim 1, wherein
a ratio of carbon to fluorine of the fluorocarbon is equal to or larger than 0.75.

3. The method according to claim 1, wherein
the fluorocarbon gas includes the fluorocarbon which has the main chain of six or more carbons bonded in the linear manner, the main chain having the structure of single bond and double bond alternately joined, and
the fluorocarbon has, regarding carbon and fluorine, a composition represented by a general formula: Cₓ₁F_{y1}, wherein x1 and y1 are numbers satisfying x1≥6 and y1≥x1+2.

4. The method according to claim 1, wherein
the fluorocarbon has at least one structure selected from the group consisting of a structural formula (1) and a structural formula (2) below.

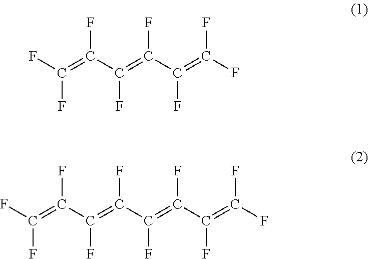 [SMILES: FC(F)=C(F)/C(F)=C(\F)C(F)=C(F)F ; FC(F)=C(F)/C(F)=C(F)/C(F)=C(\F)C(F)=C(F)F]

5. The method according to claim 1, wherein
the fluorocarbon gas includes the fluorocarbon which has the main chain of six or more carbons bonded in the linear manner, the main chain having the structure of single bond and triple bond alternately joined, and
the fluorocarbon has, regarding carbon and fluorine, a composition represented by a general formula: Cₓ₂F₂, wherein x2 and y2 are numbers satisfying x2≥6 and y2≥6.

6. The method according to claim 1, wherein
the fluorocarbon has at least one structure selected from the group consisting of a structural formula (3) and a structural formula (4) below.

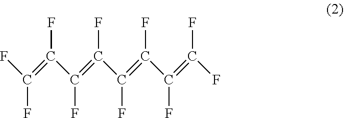 [SMILES: FC(F)=C(F)/C(F)=C(F)/C(F)=C(\F)C(F)=C(F)F]

7. The method according to claim 1, further comprising:
vaporizing the fluorocarbon being a liquid raw material to obtain a vaporized component; and
generating the plasma of the fluorocarbon gas which includes the vaporized component of the fluorocarbon.

8. The method according to claim 1, wherein:
the silicon-containing film has a silicon oxide film or a stacked film of a silicon oxide film and a silicon nitride film formed above a semiconductor substrate; and
the etching of the silicon-containing film includes: forming an etching mask having an opening on the silicon-containing film; disposing the semiconductor substrate having the silicon-containing film and the etching mask in a chamber; and forming a hole in accordance with the opening in the silicon-containing film by using the plasma of the fluorocarbon gas generated in the chamber.

9. The method according to claim 8, wherein
the plasma is generated by applying a high-frequency voltage between a first electrode which is disposed in the chamber and on which the semiconductor substrate is mounted, and a second electrode disposed in the chamber so as to face the first electrode.

10. A plasma etching method, comprising:
etching a silicon-containing film by using plasma of a fluorocarbon gas, wherein
the fluorocarbon gas includes a fluorocarbon which has a main chain of five or more carbons bonded in a linear manner, the main chain having a structure which includes double bond and triple bond.

11. The method according to claim 10, wherein
a ratio of carbon to fluorine of the fluorocarbon is equal to or larger than 0.75.

12. The method according to claim 10, wherein
the fluorocarbon has, regarding carbon and fluorine, a composition represented by a general formula: Cₓ₃F_{y3}, wherein x3 and y3 are numbers satisfying x3≥5 and y3≥6.

13. The method according to claim 10, wherein
the fluorocarbon has at least one structure selected from the group consisting of a structural formula (5) and a structural formula (6) below.

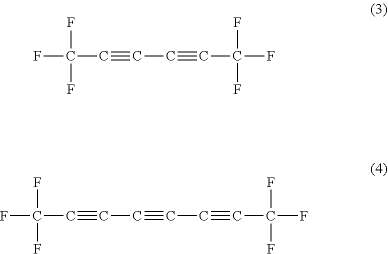 [SMILES: FC(F)(F)C#CC#CC(F)(F)F ; FC(F)(F)C#CC#CC#CC(F)(F)F]

14. The method according to claim 10, further comprising:
vaporizing the fluorocarbon being a liquid raw material to obtain a vaporized component; and
generating the plasma of the fluorocarbon gas which includes the vaporized component of the fluorocarbon.

15. The method according to claim 10, wherein:
the silicon-containing film has a silicon oxide film or a stacked film of a silicon oxide film and a silicon nitride film formed above a semiconductor substrate; and
the etching of the silicon-containing film includes: forming an etching mask having an opening on the silicon-containing film; disposing the semiconductor substrate having the silicon-containing film and the etching mask in a chamber; and forming a hole in accordance with the opening in the silicon-containing film by using the plasma of the fluorocarbon gas generated in the chamber.

16. The method according to claim 15, wherein
the plasma is generated by applying a high-frequency voltage between a first electrode which is disposed in the chamber and on which the semiconductor substrate is mounted, and a second electrode disposed in the chamber so as to face the first electrode.

17. A plasma etching apparatus, comprising:
a chamber in which a substrate to be subjected to etching is disposed;
an electrode disposed in the chamber;
a first process gas introduction unit comprising: a raw material tank accommodating a liquid raw material which includes at least one selected from the group consisting of a first fluorocarbon which has a first main chain of six or more carbons bonded in a linear manner, the first main chain having a structure of single bond and double bond alternately joined, a second fluorocarbon which has a second main chain of six or more carbons bonded in a linear manner, the second main chain having a structure of single bond and triple bond alternately joined, and a third fluorocarbon which has a third main chain of five or more carbons bonded in a linear manner, the third main chain having a structure which includes double bond and triple bond; and a vaporization mechanism vaporizing the liquid raw material so as to introduce a fluorocarbon gas which includes a vaporized component of the liquid raw material into the chamber; and
a power supply applying a voltage to generate plasma of the fluorocarbon gas to the electrode.

18. The apparatus according to claim 17, wherein
the vaporization mechanism comprises a vaporizer which is provided in a pipe connecting the raw material tank and the chamber and vaporizes the liquid raw material, and
the first process gas introduction unit further comprises: a liquid flow rate controller which is provided at a position between the raw material tank and the vaporizer in the pipe and controls a flow rate of the liquid raw material; and a heater provided in a periphery of a portion positioned between the vaporizer and the chamber in the pipe.

19. The apparatus according to claim 17, wherein
the vaporization mechanism comprises a first heater which is provided in a periphery of the raw material tank and vaporizes the liquid raw material in the raw material tank, and
the first process gas introduction unit further comprises: a gas flow rate controller which is provided in a pipe connecting the raw material tank and the chamber and controls a flow rate of the vaporized component of the liquid raw material; and a second heater provided in a periphery of the pipe.

20. The apparatus according to claim 17, further comprising:
a second process gas introduction unit configured to introduce a process gas which includes at least one selected from the group consisting of He gas, Ar gas, Kr gas, Xe gas, N₂ gas, O₂ gas, H₂ gas, CO gas, NF₃ gas, SF₆ gas, CH₄ gas, CF₄ gas, C₄F₆ gas, C₄F₈ gas, CHF₃ gas, CH₂F₂ gas, and CH₃F gas, into the chamber.
